(19) United States
(12) Patent Application Publication
(10) Pub. No.: US 2004/0106998 A1
(43) Pub. Date: Jun. 3, 2004

(54) Multiaxial artificial disc replacements

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/679,667

(22) Filed: Oct. 6, 2003

Related U.S. Application Data
(60) Provisional application No. 60/416,181, filed on Oct. 4, 2002.

(51) Int. Cl. A61F 2/44

(52) U.S. Cl. 623/17.16; 623/17.11

(57) ABSTRACT

An artificial disc replacement (ADR) that pivots along multiple, independent axes to accommodate both flexion/extension and lateral bending. In the preferred embodiment, the mechanism is a cruciate-shaped axle that allows independent movement along orthogonal axes, much like a “universal joint.” The ADR can be used as a standalone device that attaches to the vertebrae, or can be mobile and tethered by way of a “link member.” Friction between the axle and the top and bottom components can be reduced with needle-like roller bearings. Cushioning material, such as elastomerics, hydrogels, and other compressible resilient materials or springs may be used to control movement.

### REFERENCE TO RELATED APPLICATION

This application claims priority from U.S. Provisional Patent Application Serial No. 60/416,181, filed Oct. 4, 2002, the entire content of which is incorporated herein by reference.

### FIELD OF THE INVENTION

This invention relates generally to artificial intervertebral disc replacements and, in particular, to a multiaxial ADR.

### BACKGROUND OF THE INVENTION

Many spinal conditions, including degenerative disc disease, can be treated by spinal fusion or through artificial disc replacement (ADR). Since spinal fusion eliminates motion across fused segments of the spine, the discs adjacent to the fused level are subjected to increased stress. The increased stress increases the changes of future surgery to treat the degeneration of the discs adjacent to the fusion.

ADRs offer several advantages over spinal fusion, the most important of which is the preservation of spinal motion. One of the most important features of an artificial disc replacement (ADR) is its ability to replicate the kinematics of a natural disc. ADRs that replicate the kinematics of a normal disc are less likely to transfer additional forces above and below the replaced disc. In addition, ADRs with natural kinematics are less likely to stress the facet joints and the annulus fibrosus (AF) at the level of the disc replacement. Replicating the movements of the natural disc also decreases the risk of separation of the ADR from the vertebrae above and below the ADR.

The kinematics of ADRs are governed by the range of motion (ROM), the location of the center of rotation (COR) and the presence (or absence) of a variable center of rotation (VCOR). Generally ROM is limited by the facet joints and the AF. A natural disc has a VCOR, that is, the COR varies as the spine bends forward (flexion) and backward (extension). Typically, the vertebra above a natural disc translates forward 1-2 mm as the spine is flexed.

Prior art total disc replacements (TDR), that is, ADRs with rigid plates that attach to the vertebrae, do not replicate the kinematics of the natural disc. Most prior art TDRs also rely on a single, fixed COR. As a result, many of the prior art TDRs have a limited ROM. Although there does exist ADR devices with a single hinge joint that allows for flexion and extension, the need remains for an ADR that facilitates movement along multiple, independent axes to accommodate both flexion/extension and lateral bending.

### SUMMARY OF THE INVENTION

This invention improves upon the existing art by providing an artificial disc replacement (ADR) that pivots along multiple, independent axes to accommodate both flexion/extension and lateral bending. In the preferred embodiment, a cruciate-shaped axle is provided to allow independent movement along orthogonal axes, much like a “universal joint.” The ADR can be used as a standalone device that attaches to the vertebrae, or can be mobile an attached by way of a link member.

The invention offers many advantages. In addition to a robust design requiring few essential components, the dimensions of the top and bottom components may be adjusted to determine the allowed range of motion. For example, the components can be sized to impinge at 5 degrees of extension and lateral bending. Limiting motions in these directions may be important to avoid excessive pressure on the facet joints.

Needle roller bearings can be used to reduce the friction between the axle and the components. Cushioning material, such as elastomerics, hydrogels, and other compressible resilient materials or springs may be used to control movement.

### BRIEF DESCRIPTION OF THE DRAWINGS

FIG. 1 shows an anterior view of the spine and a multiaxial device according to the present invention;

FIG. 2 is a lateral view of the spine and the ADR shown in FIG. 1;

FIG. 3A is a view of the superior surface of the bottom component with the axle in place;

FIG. 3B is a view of the inferior surface of the top component and a locking clip that holds the top component in place;

FIG. 4 is a view of the cruciate-shaped axle;

FIG. 5A is a view of the superior surface of the bottom component, axle, projected location of the axle coupling section of the top component;

FIG. 5B is a view of the device drawn in FIG. 5A;

FIG. 6 is a view of the lateral surface of the device with springs;

FIG. 7A shows the lateral view of an alternative embodiment of the axle of the present invention;

FIG. 7B is a view of the anterior aspect of the device shown in FIG. 7A;

FIG. 8A shows the view of an alternative embodiment of the endplate of the present invention;

FIG. 8B shows the view of the axle side of the embodiment of the shown in FIG. 8A;

FIG. 8C shows the lateral view of the endplates shown in FIGS. 8A and 8B;

FIG. 9 shows a lateral view of the embodiments of the device shown in FIGS. 8A-8C; and

FIG. 10 is a view of the anterior, or lateral, aspect of the device shown in FIG. 9.

### DETAILED DESCRIPTION OF THE INVENTION

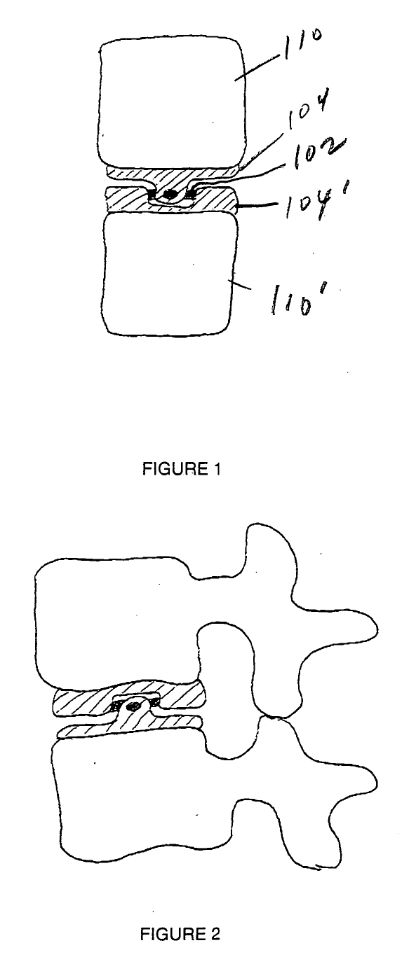

FIG. 1 is an anterior view of the spine and a multiaxial device according to the invention disposed between upper and lower vertebral bodies 110, 110′. The area 102 represents a cruciate-shaped axle. The top and bottom components 104, 104′ rotate around the four arms of the axle.

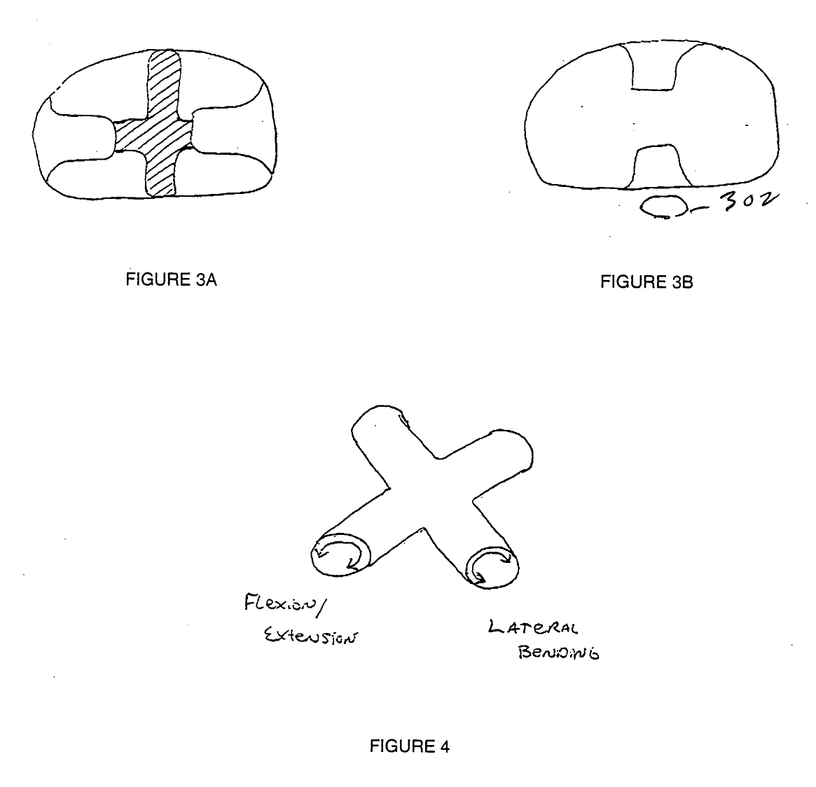

FIG. 2 is a lateral view of the spine and the ADR drawn in FIG. 1. FIG. 3A is a view of the superior surface of the bottom component with the axle 102 in place. The bottom component rotates around the axle to allow flexion and extension of the spine. FIG. 3B is a view of the inferior surface of the top component and a locking clip 302 that holds the top component in place.

FIG. 4 is a view of the cruciate-shaped axle. In this particular embodiment, rotation about anterior and posterior arms of the axle permits approximately 5 degrees of lateral bending to the left or right. Rotation about the left and right arms of the axle permits approximately 5 degrees of spinal extension and about 15 degrees of spinal flexion. The extent of such movements may be varied according to the invention through appropriate adjustment to geometrical dimensions.

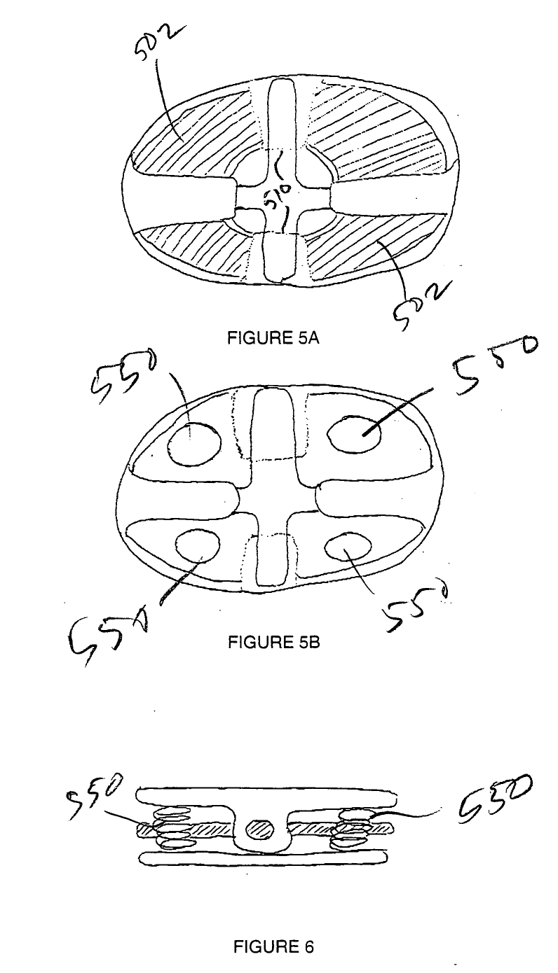

FIG. 5A is a view of the superior surface of the bottom component, axle, projected location of the axle coupling section of the top component, and a cushioning material 502 such as an elastomer. The area 510 represents the projected location of the axle coupling section of the top component. The elastomer, which positioned between the components, may be glued to either the top or bottom component but preferably not to both.

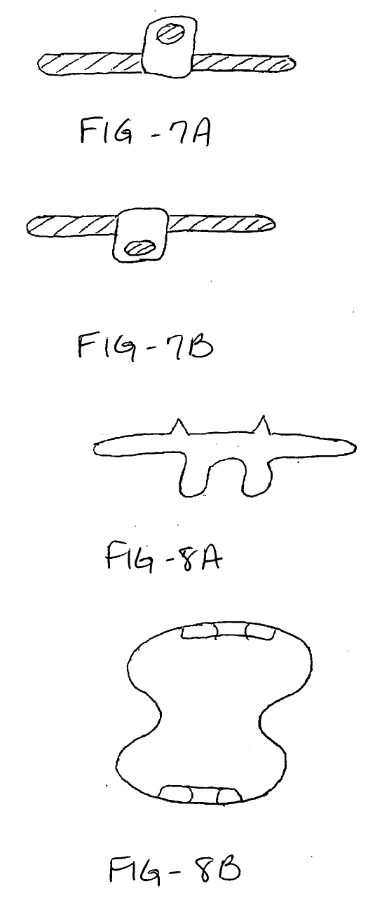

FIG. 5B is a view of an alternative embodiment wherein the elastomer has been replaced by springs 550. FIG. 6 is a view of the lateral surface of the device with springs. To better illustrate the springs, the coupling portion of the bottom component is not drawn. FIG. 7A is the lateral view of an alternative embodiment of an axle according to the invention. FIG. 7B is a view of the anterior aspect of the device shown in FIG. 7A. Again, the two axles are coupled at 90 degrees to one another with an additional component.

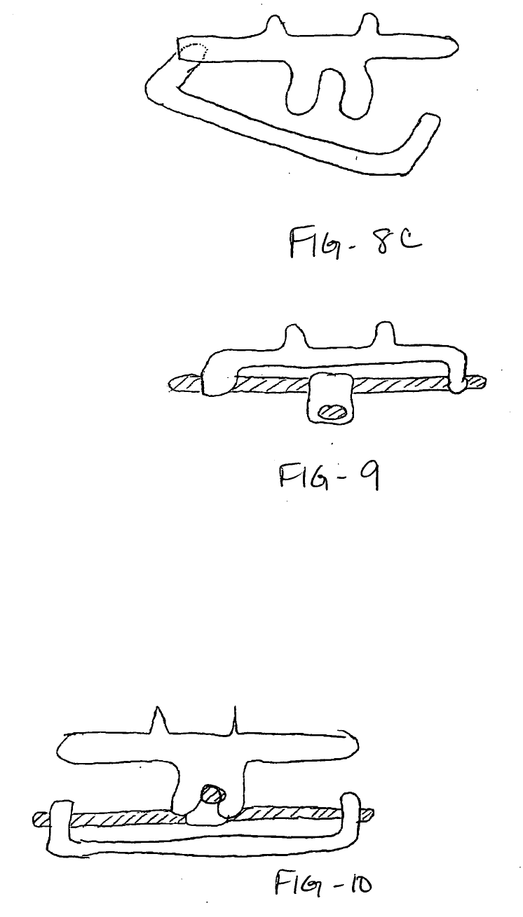

FIG. 8A is the view of an alternative embodiment of an endplate according to the invention that captures the axles in a manner that allows the axles to piston up and down. This less constrained embodiment should help prevent the shear stress at the vertebra-endplate surface.

FIG. 8B is the view of the axle side of the embodiment of the ADR endplate shown in FIG. 8A. The sides of the endplate can be sculpted to allow greater range of motion between the endplates.

FIG. 8C is a lateral view of the endplates shown in FIGS. 8A and 8B. The raised portion of one endplate can rotate into the sculpted space of the second endplate without impinging on the second endplate.

FIG. 9 is a lateral view of the embodiments of the device described above. The axles can be lengthened to allow one vertebra to slide relative to the second vertebra. Optional translocation stops could be added to the axles. For example, the axles could have enlarged areas that limit the amount of translocation of one vertebra relative to the other. FIG. 10 is a view of the anterior, or lateral, aspect of the device shown in FIG. 9.

In all embodiments, the top and bottom components may include ingrowth surfaces for use as a stand-alone device. Alternatively, top and bottom components can have polished surfaces for use with a “mobile link” of the type described, for example, in my co-pending U.S. patent application Ser. No. 10/426,995, the entire content of which is incorporated herein by reference.

A seal could be used to trap debris inside the ADR. For example, the seal may surround the periphery of the superior ADR EP and the inferior ADR EP. Alternatively, the seal could be placed around the central articulating component. The seal could also hold a fluid within the ADR. Various fluids including: water or aqueous solutions, triglyceride oil, soybean oil, an inorganic oil (e.g. silicone oil or fluorocarbon), glycerin, ethylene glycol, or other animal, vegetable, synthetic oil, or combinations thereof could be used. The seal could be made of an expandable elastomer such as those used in medical devices for the cardiovascular system.

We claim:

1. A multiaxial artificial disc replacement (ADR), comprising:
a lower component adapted for fixation to an inferior vertebral body;
an upper component adapted for fixation to a superior vertebral body; and
an element that allows movement between the lower and upper components along two separate, independent axes.

2. The multiaxial ADR of claim 1, wherein the two axes are orthogonal to one another.

3. The multiaxial ADR of claim 1, wherein one of the axes is generally medial-lateral, and the other axis is generally anterior-posterior.

4. The multiaxial ADR of claim 1, wherein the element that allows movement between the lower and upper components is a cruciate-shaped axle.

5. The multiaxial ADR of claim 1, further including a cushioning component situated between the upper and lower components.

6. The multiaxial ADR of claim 5, wherein the cushioning component is an elastomer.

7. The multiaxial ADR of claim 5, wherein the cushioning component is a hydrogel.

8. The multiaxial ADR of claim 5, wherein the cushioning component is a spring.

9. The multiaxial ADR of claim 1, wherein one or both of the upper and lower components include a bone-ingrowth surface associated with fixation.

10. The multiaxial ADR of claim 1, wherein one or both of the upper and lower components are loosely coupled to a respective vertebral body with a mobile link.

11. The multiaxial ADR of claim 1, further including a seal to contain debris.

12. The multiaxial ADR of claim 11, wherein the seal surrounds the periphery of the upper and lower components.

13. The multiaxial ADR of claim 11, wherein the seal is disposed around the central articulating component.

14. The multiaxial ADR of claim 1, further including a fluid-containing seal.

15. The multiaxial ADR of claim 1, wherein the fluid includes water or aqueous solutions, triglyceride oil, soybean oil, an inorganic oil (e.g. silicone oil or fluorocarbon), glycerin, ethylene glycol, or other animal, vegetable, synthetic oil, or combinations thereof.
